(19) United States
(12) Patent Application Publication
(10) Pub. No.: US 2024/0122871 A1
(43) Pub. Date: Apr. 18, 2024

(54) TRANSDERMAL CANNABINOID TEMPORARY TATTOO

(71) Applicant: Nicole Maria Furrer, Saint Louis, MO (US)

(72) Inventor: Nicole Maria Furrer, Saint Louis, MO (US)

(21) Appl. No.: 18/492,164

(22) Filed: Oct. 23, 2023

Related U.S. Application Data
(60) Provisional application No. 63/417,209, filed on Oct. 18, 2022.

(51) Int. Cl. A61K 9/70; A61K 31/00; A61K 47/54

(52) U.S. Cl. CPC A61K 9/7076; A61K 31/658; A61K 47/542

(57) ABSTRACT

A drug-in-adhesive device for transdermal delivery of cannabinoids to the bloodstream utilizes a decal-style temporary tattoo as the backing layer. The temporary tattoo is printed with colors and designs to create an aesthetically pleasing appearance that is more attractive than a typical transdermal patch backing layer. The tattoo includes a water-based drug-in-adhesive compound sandwiched between a release liner and a backing layer made from water release transfer paper with an inkjet-printed design. When the drug-in-adhesive compound adheres to the skin, the cannabinoids diffuse from the adhesive compound through the stratum corneum layer of the skin, through the dermis, and into the microvascular system of the body.

### CROSS-REFERENCE TO RELATED APPLICATION

This application claims the benefit of priority of U.S. provisional application No. 63/417,209, filed Oct. 22, 2022, the contents of which are herein incorporated by reference.

### BACKGROUND OF THE INVENTION

The present invention relates to transdermal cannabinoid delivery devices and, more particularly, to a transdermal temporary tattoo device for delivering precise dosages of cannabinoids to the user's bloodstream.

Cannabis refers to various strains of plants Cannabis sativa or Cannabis indica. The cannabis plant produces an estimated 80+ cannabinoids, each of which has unique pharmacologic effects. Cannabinoids are a class of diverse chemical compounds that act on cannabinoid receptors on cells that affect neurotransmitter release in the brain. Tetrahydrocannabinol (THC) is the primary psychoactive compound of cannabis.

The medicinal benefits of cannabinoids are well known. But the most common methods of administration—smoking, vaping, and eating—are not the most effective or safest methods of the administration of cannabinoids. Smoking and vaping both pose risks to the lungs, while eating cannabinoids is a less effective method of administration due to the first pass of the digestive system.

Inhalation delivery methods of smoking and vaporization of cannabinoids have no reliable dosage as medicine. Bioavailability of the smoking route was reported as anywhere from 2-56%. The number, duration, and spacing of puffs, hold time, and inhalation volume greatly influences the degree of exposure and blood levels. Oral delivery methods of ingesting extracts, infusions and edibles forms of cannabinoids have typically a delay in the onset of their actions making it extremely difficult to ingest an exact, predictable dosage of cannabinoids. The oral absorption of THC and cannabidiol (CBD) is typically reported as 6% bioavailability after extensive first pass liver metabolism. Oral delivery is slow and unpredictable, with peak concentrations occurring 1-5 hours post-close. THC is rapidly metabolized in the body, such that concentration levels of the chemical in the bloodstream decrease rapidly if administered through traditional methods.

In contrast, a transdermal application allows for small dosages of THC to be administered over an extended period of time, thereby allowing the concentration levels of the chemical to remain relatively steady in the bloodstream. The transdermal administration of cannabinoids provides many advantages over other administration routes like smoking or eating cannabis edibles. Transdermal cannabinoids enter the bloodstream faster than edible cannabinoids and pose no risks to the lungs.

Transdermal delivery devices are a common mode of administration for many different types of drugs, including cannabinoids. See, for example, US20120310187A1, US20220160647A1, US20200338041A1, and U.S. Pat. No. 6,113,940A, the disclosures of which are incorporated by reference in their entireties. Transdermal cannabinoid delivery devices utilize a backing layer, a pressure sensitive adhesive, and a release liner. Pressure sensitive adhesive is a type of nonreactive adhesive which forms a bond when pressure is applied to bond the adhesive with a surface. No solvent, water, or heat is needed to activate the adhesive. The transdermal cannabinoid delivery devices, such as matrix patches, utilize a backing layer on the surface of the skin to adhere the drug-in-adhesive to the body. These backing layers look like “flesh” colored bandages when applied to the skin and remain on the skin until the device is removed. The problem is that the flesh color does not work for all skin tones, nor is it aesthetically attractive.

Decal-style temporary tattoos are a popular method of decorating the skin. Prior art temporary tattoos utilize a backing layer, a pressure sensitive adhesive, and a release liner. They are made by printing an image onto a paper backing layer coated with a transfer film. See U.S. Pat. Nos. 6,042,881A and 4,594,276A, for example, the disclosures of which are incorporated by reference in their entireties. To apply the tattoo, the user removes the release liner, applies the tattoo to the skin adhesive side down, and then moistens the paper which releases from the transfer film that holds the adhesive to the skin. The temporary tattoo generally remains adhered to the skin for 24-48 hours.

As can be seen, there is a need for a temporary tattoo transdermal cannabinoid delivery device with a backing layer that is removed from a pressure sensitive drug-in-adhesive upon application.

### SUMMARY OF THE INVENTION

In one aspect of the present invention, a temporary tattoo for transdermal delivery of cannabinoids comprises a release liner; a drug-in-adhesive composition releasably affixed to the release liner, comprising a nanoemulsion of at least one cannabinoid; and a water release transfer paper backing printed with a tattoo design affixed to the drug-in-adhesive composition.

The decal-style temporary tattoo provides transdermal delivery of cannabinoids to the bloodstream. The temporary tattoo may be inkjet printed with colors and designs comprising non-toxic, skin-safe ink to create an aesthetically pleasing appearance that is more attractive than a typical transdermal patch backing layer. The temporary tattoo may be provided in a variety of sizes.

Embodiments of the present invention may comprise one or more cannabinoids, including but not limited to THC, CBD, cannabinol (CBN), tetrahydrocannabinolic acid (THCA), and Cannabidiolic acid (CBDA).

A method according to another embodiment of the present invention includes printing skin-safe ink designs containing cannabinoids and colors on water release paper with an inkjet printer.

These and other features, aspects and advantages of the present invention will become better understood with reference to the following drawings, description, and claims.

### BRIEF DESCRIPTION OF THE DRAWINGS

FIG. 1 is a front view of a transdermal cannabinoid-infused temporary tattoo, according to an embodiment of the present invention;

FIG. 2 is another front view thereof;

FIG. 3 is an exploded view thereof;

FIG. 4 is a perspective view thereof, shown in use; and

FIG. 5 is a flow chart of a method of use according to an embodiment of the present invention, including a step of preparing the transdermal temporary tattoo of FIG. 1.

### DETAILED DESCRIPTION OF THE INVENTION

The following detailed description is exemplary in nature of the best currently contemplated modes of carrying out exemplary embodiments of the invention. The description is not intended to limit the scope, applicability, or configuration of the invention in any way but is made merely for the purpose of illustrating the general principles of the invention, since the scope of the invention is best defined by the appended claims. Rather, the following description provides some practical illustrations for implementing exemplary embodiments of the present invention.

Broadly, one embodiment of the present invention is a temporary tattoo article for transdermal delivery of cannabinoids to the bloodstream of the user, comprising a water-based drug-in-adhesive composition sandwiched between a release liner layer and a backing layer made from water release transfer paper with an inkjet-printed design. When the drug-in-adhesive composition adheres to the skin, the cannabinoids diffuse from the adhesive composition through the stratum corneum layer of the skin, through the dermis and into the microvascular system of the body to the bloodstream.

Cannabinoids as used herein refers to any cannabinoid, also defined as any ligand of the cannabinoid receptor and related compounds. Cannabinoids include phytocannabinoids (obtained from plants). Most phytocannabinoids fall into subclasses such as cannabigerol, cannabichromene, cannabidiol, and cannabinol (including tetrahydrocannabinol, e.g., Δ9-THC, Δ8-THC, etc.).

As used herein, the term “drug-in-adhesive” refers to an adhesive layer of a transdermal patch which contains the drug to be released.

A water-based drug-in-adhesive composition according to an embodiment of the present invention comprises a nanoemulsion of cannabinoids, i.e., a single cannabinoid or a mixture of cannabinoid (e.g., one or more of THC, CBD, CBN, etc.), a skin penetration enhancer, and a water-based, pressure sensitive skin safe adhesive. An emulsion is a liquid-liquid dispersion wherein a first liquid acts as a continuous medium, and a second liquid acts as a dispersion medium of droplets in the first liquid. Emulsions with nanoscopic droplet sizes (typically in the range of 20-200 nm) are referred to as nanoemulsions. An emulsion (including nanoemulsions) can be of two types: a water-in-oil emulsion or an oil-in-water emulsion. For a water-in-oil emulsion, oil is the continuous phase, and water is the dispersed phase. For an oil-in-water emulsion, water is the continuous phase, and oil is the dispersed phase. See U.S. Ser. No. 10/738,268B2 and WO2020081550A1, the disclosures of which are incorporated by reference in their entireties.

The composition may include an amount of adhesive generally between about 50% and about 80% by weight of the total composition, dependent upon the cannabinoid content in the cannabis nano emulsion formulation. Compositions having lower amounts of the selected cannabinoid(s) have a higher amount of adhesive. The cannabinoid emulsion and adhesive are blended together in a ratio that delivers a selected dosage to each tattoo in the manufacturing process. For example, the cannabis emulsion and adhesive mixture may include THC in a ratio of 10 to 20 milligrams per tattoo. Preferably, the skin adhesive composition contains a plurality of cannabinoids. In some cases, the mixture may include 15 to 20 milligrams of a combination of THC, CBD, CBN and other cannabinoids.

The nanoemulsion formulation disclosed herein comprises an oil phase dispersed in an aqueous phase. Thus, the nanoemulsion formulation is an oil-in-water emulsion.

The oil phase of the nanoemulsion formulation comprises at least one cannabinoid and a skin permeation enhancer, such as oleic acid. The skin penetration enhancer is added to increase the body's absorption of cannabinoids. The cannabinoid may be present in the nanoemulsion formulation in a weight ratio of from about 0.1% to about 15%. In embodiments having more than one cannabinoid, the recited weight ratios for the cannabinoid represent a total weight ratio for all cannabinoids present in the nanoemulsion formulation.

The aqueous phase of the nanoemulsion formulation comprises water and optionally additional components. The aqueous phase is present in the nanoemulsion formulation in a weight ratio of between about 50% and about 90%.

In some embodiments, the composition comprises a coloring agent, such as an inkjet ink. While commonly used inkjet ink is not considered safe for consumption by the US Food and Drug Administration (FDA), vegetable-based inkjet ink is FDA approved for consumption, making it safe to use on the human body. Therefore, a vegetable-based inkjet ink is preferred. Reference is made to AU2014230931B2, the disclosure of which is incorporated by reference in its entirety.

The cannabinoid nanoemulsion may be manufactured by homogenizing the cannabis oils, penetration enhancer, carrier oil, surfactant, and water with an ultrasonic homogenizer. This process breaks down the cannabis oils into nano-sized particles, suspended in the emulsion, that are small enough to penetrate the skin and enter the bloodstream of the user. The adhesive is in a liquid state at the beginning of the manufacturing process, which allows the water-based cannabinoid nanoemulsion to fully disperse into the water-based adhesive.

Inkjet printing technology is a reliable, quick, and convenient method of printing digital images on a variety of surfaces. One of the advantages of inkjet printing is that the printed image can be varied simply by sending a different electronic signal to the print-head, allowing different images to be printed and so producing products with visual variety.

The temporary tattoo device comprises a backing layer made from water release transfer paper. Water release transfer paper is known in the art of temporary tattoo manufacturing as temporary tattoo paper.

To manufacture a temporary tattoo according to an embodiment of the present invention, a sizing agent is applied to a paper substrate, which modifies the paper's stiffness and texture. A layer of non-stick release coating is then applied to the paper, to help the image separate from the backing paper. A transfer film may then be coated on top of the silicone layer. This film is the layer that the image is printed upon. These materials are designed to be strong enough to adhere to the backing paper during printing yet flexible enough to be easily released during application.

Designs may have any color or combination of colors, and any size. In one embodiment the tattoo design is a cannabis plant leaf, printed, for example, in black ink.

A coating of the water-based adhesive composition is spread over a sheet of release liner. When the adhesive composition dries to a sticky solid, a sheet of inkjet printed water transfer tattoo paper is applied over the dried adhesive layer on the release liner, creating a sandwich of adhesive composition between the release liner and the tattoo paper.

The temporary tattoo product may be any suitable size. In an example, the tattoo may be 5 inches in length and 1 inch in width.

Embodiments of the temporary tattoo may have a variety of closes. The number of milligrams of cannabinoids in any temporary tattoo disclosed herein is determined by the size of the water release paper, as well as the concentration of cannabinoids in the adhesive composition.

The temporary tattoo may comprise a silicone-based release liner. The release liner serves to protect the drug-in-adhesive layer until the user is ready to apply the temporary tattoo. Immediately prior to application to the skin, the release liner is removed from the temporary tattoo so that the adhesive may be adhered directly to the user's skin while the film layer from the water release paper protects the adhesive from the external side of the temporary tattoo. The release liner may be made of any material known in the art that may be removed from the backing cleanly, with minimal displacement of the adhesive-cannabinoid composition therebetween.

To use the temporary tattoo, the user removes the release liner and applies the adhesive surface of the temporary tattoo to the skin. Then the user applies water and pressure to the water release backing paper, which releases the backing paper from the thin film on which the image is printed, leaving the temporary tattoo adhered on the skin. Upon drying on the skin, the film adheres tightly and smoothly to maintain the image quality. The temporary tattoo remains adhered to the skin for up to 48 hours. The skin allows transdermal permeation of nano-sized molecules, so the cannabinoids avoid the digestive system and go straight to the bloodstream of the user, a faster and more effective application than edible cannabinoids.

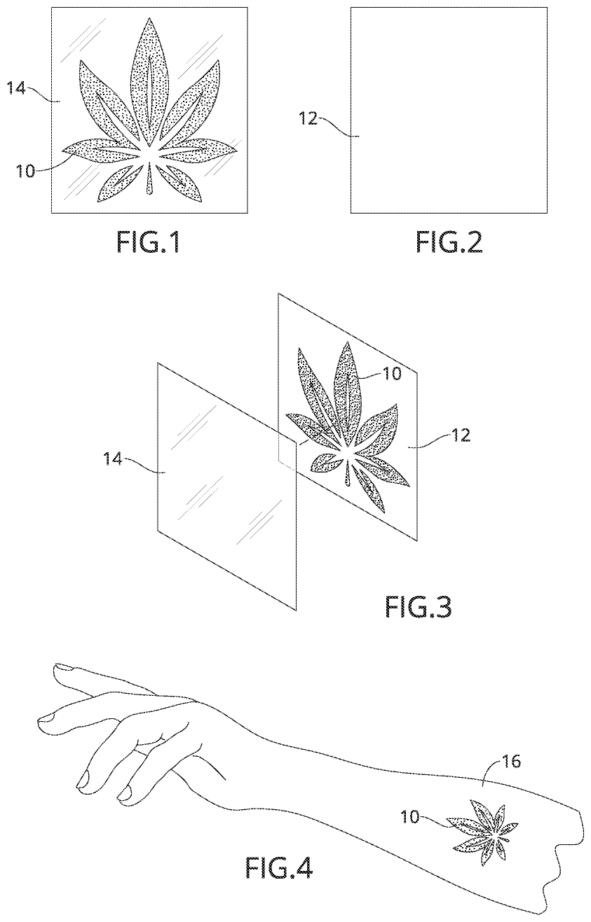

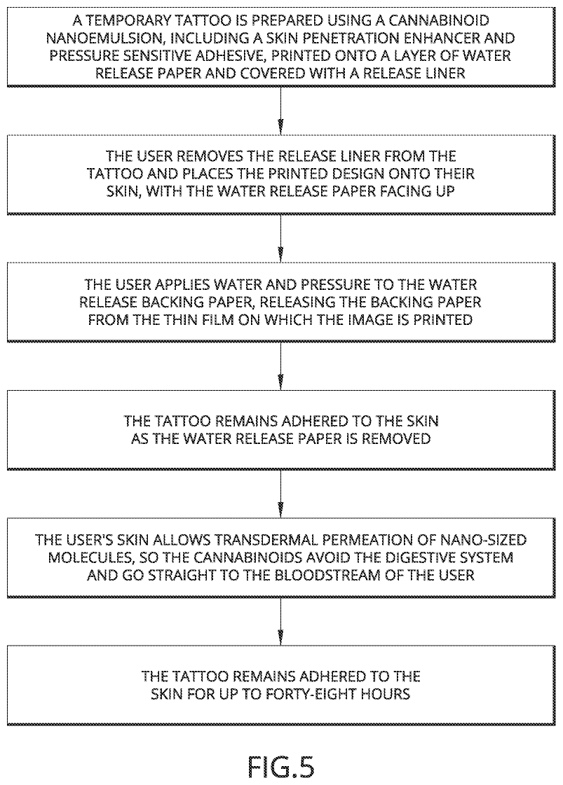

Referring to FIGS. 1 through 5, a temporary tattoo 10 containing cannabinoids in an adhesive is shown in FIG. 1, configured as a graphic of a marijuana leaf. The temporary tattoo 10 is shown with a protective liner 14. As shown in FIGS. 2 and 3, the temporary tattoo 10 is mounted on a release coated backing paper 12. FIG. 4 illustrates the temporary tattoo 10 applied to a user's 16 skin, enabling transdermal administration of the cannabinoids contained therein.

FIG. 5 shows a flowchart beginning with a summary of a method of manufacturing the temporary tattoo of FIG. 1, combining a cannabinoid nanoemulsion, a skin penetration enhancer, and a pressure sensitive adhesive, printing the mixture onto a backing, and applying a protective liner. A method of using the temporary tattoo includes removing the liner, placing the adhesive layer on the user's skin, and applying water to the backing to remove the backing from the temporary tattoo. Once the temporary tattoo is adhered to the skin, it releases cannabinoids for absorption through the skin. While the temporary tattoo may be worn for any suitable period, it may be removed, such as up to 48 hours after application.

It should be understood, of course, that the foregoing relates to exemplary embodiments of the invention and that modifications may be made without departing from the spirit and scope of the invention as set forth in the following claims.

What is claimed is:

1. A temporary tattoo for transdermal delivery of cannabinoids, comprising:
a release liner;
a drug-in-adhesive composition releasably affixed to the release liner, comprising a nanoemulsion of at least one cannabinoid; and
a water release transfer backing paper printed with a tattoo design affixed to the drug-in-adhesive composition.

2. The temporary tattoo of claim 1, wherein the tattoo design comprises a vegetable-based inkjet ink.

3. The temporary tattoo of claim 1, wherein the at least one cannabinoid is present in a quantity containing at least about 10 mg cannabinoids.

4. The temporary tattoo of claim 1, wherein the at least one cannabinoid contains a plurality of cannabinoids.

5. The temporary tattoo of claim 1, wherein the at least one cannabinoid is about 0.1 weight % to about 15 weight % of a total of the drug-in-adhesive composition.

6. The temporary tattoo of claim 1, wherein the at least one cannabinoid is about 0.1 weight % to about 15 weight % of the nanoemulsion.

7. The temporary tattoo of claim 1, wherein about 50 weight % to about 90 weight % of the nanoemulsion is an aqueous phase.

8. The temporary tattoo of claim 1, wherein the nanoemulsion further comprises a carrier oil and a surfactant.

9. The temporary tattoo of claim 1, wherein said drug-in-adhesive composition further comprises a water-based, pressure sensitive adhesive and a skin penetration enhancer.

10. The temporary tattoo of claim 9, wherein the water-based, pressure sensitive adhesive is about 50 weight % to about 80 weight % of a total of the drug-in-adhesive composition.

11. The temporary tattoo of claim 9, wherein the skin penetration enhancer is oleic acid.
